(19) United States
(12) Patent Application Publication
(10) Pub. No.: US 2014/0283156 A1
(43) Pub. Date: Sep. 18, 2014

(54) TRANS-SPLICING RIBOZYMES AND SILENT RECOMBINASES

(71) Applicants: Anthony M. Zador, Cold Spring Harbor, NY (US); Ian D. Peikon, Cold Spring Harbor, NY (US)

(72) Inventors: Anthony M. Zador, Cold Spring Harbor, NY (US); Ian D. Peikon, Cold Spring Harbor, NY (US)

(73) Assignee: COLD SPRING HARBOR LABORATORY, Cold Spring Harbor, NY (US)

(21) Appl. No.: 14/208,432

(22) Filed: Mar. 13, 2014

Related U.S. Application Data
(60) Provisional application No. 61/782,533, filed on Mar. 14, 2013.

(51) Int. Cl. C12N 15/113; C12N 15/90

(52) U.S. Cl. CPC C12N 15/113; C12N 15/907
USPC 800/13; 435/320.1; 435/325; 435/462; 536/24.1; 435/456

(57) ABSTRACT

The present invention provides a trans-splicing ribozyme comprising i) a targeting nucleotide sequence that is complementary to a target nucleotide sequence within a mRNA that is expressed in a cell; contiguous with ii) a catalytic RNA sequence; contiguous with iii) a donor transcript, which donor transcript comprises at least a nucleotide sequence that encodes a trans-activator, wherein when the trans-splicing ribozyme is expressed in a cell, the catalytic RNA sequence cleaves the mRNA and ligates the donor transcript to the mRNA to generate a spliced mRNA which comprises the donor transcript, such that the donor transcript is translated as part of the spliced mRNA in the cell, as well as methods of using the trans-splicing ribozyme. The present invention also provides variants of Cre and other recombinases, as well as method of using the variants.

This application claims priority of U.S. Provisional Patent Application No. 61/782,533, filed Mar. 14, 2013, the entire contents of which are hereby incorporated herein by reference.

This application incorporates-by-reference nucleotide and/or amino acid sequences which are present in the file named “140313_{—}5981_{—}80645_SEQUENCELISTING_REB.TXT”, which is 71.6 kilobytes in size, and which was created Mar. 13, 2014 in the IBM-PC machine format, having an operating system compatibility with MS-Windows, which is contained in the text file filed Mar. 13, 2014 as part of this application.

Throughout this application, various publications are referenced, including referenced in parenthesis. Full citations for publications referenced in parenthesis may be found listed at the end of the specification immediately preceding the claims. The disclosures of all referenced publications in their entireties are hereby incorporated by reference into this application in order to more fully describe the state of the art to which this invention pertains.

### BACKGROUND OF THE INVENTION

The ability to target gene expression to genetically-defined cell-types provides a powerful tool for biology and molecular therapy. Cell-type specific transgene expression can be achieved using some form of an endogenous cell-type-specific promoter. These strategies have the advantage that the transgene can be delivered virally, and therefore have the potential to work well in mammalian species. However, mammalian promoters are often very large, and the rules governing mammalian gene expression—i.e. how gene expression depends on promoters-remain poorly understood. One strategy has been to express a transgene under the control of a “minimal” endogenous promoter—a promoter short enough (<10 kb) to fit into a virus such as adeno-associated virus (AAV) or lentivirus. Unfortunately, with some notable exceptions (e.g. hypocretin), this strategy typically does not recapitulate the expression pattern of the endogenous gene. Another clever strategy involves the use of short promoters from the puffer fish (Takifugu rubripes), an organism with a much more compact genome in which the regulatory sequences are shorter. Unfortunately, fugu promoters also fail to recapitulate mammalian expression patterns. Thus at present there is no general viral strategy for delivering transgenes to genetically defined cell populations in mammals.

There is a need for a broadly applicable method for conveniently manipulating the expression of transgenes in genetically defined cell populations.

### SUMMARY OF THE INVENTION

The present invention provides a trans-splicing ribozyme comprising:
i) a targeting nucleotide sequence that is complementary to a target nucleotide sequence within a mRNA that is expressed in a cell; contiguous with
ii) a catalytic RNA sequence; contiguous with
iii) a donor transcript, which donor transcript comprises at least a nucleotide sequence that encodes a trans-activator,
   wherein when the trans-splicing ribozyme is expressed in a cell, the catalytic RNA sequence cleaves the mRNA and ligates the donor transcript to the mRNA to generate a spliced mRNA which comprises the donor transcript, such that the donor transcript is translated as part of the spliced mRNA in the cell.

The present invention provides a method of producing the trans-splicing ribozyme of the invention, comprising viral delivery of an expression vector comprising a polynucleotide encoding the trans-splicing ribozyme into a cell under conditions such that the cell expresses the trans-splicing ribozyme, thereby producing the trans-splicing ribozyme.

The present invention provides a method of expressing a recombinase-dependent transgene in a cell, comprising delivery of
a) a first expression vector which is an expression vector of the invention; and
b) a second expression vector which comprises the recombinase-dependent transgene,
   into the cell under conditions such that the cell expresses the trans-splicing ribozyme encoded in the first expression vector, and the trans-activator encoded by the trans-splicing ribozyme activates expression of the trans-activator-dependent transgene in the second expression vector, thereby expressing the trans-activator-dependent transgene in the cell.

The present invention provides a method of expressing a trans-activator-dependent transgene in a cell containing a trans-activator-dependent transgene, comprising delivery of an expression vector of the present invention into the cell under conditions such that the cell expresses the trans-splicing ribozyme encoded in the expression vector, and the trans-activator encoded by the trans-splicing ribozyme activates expression of the trans-activator-dependent transgene in the cell, thereby expressing the trans-activator-dependent transgene in the cell.

The present invention provides a Cre variant having Cre recombinase activity comprising amino acids in the sequence set forth as SEQ ID NO: 3, wherein the sequence of amino acids of the Cre variant is other than the sequence set forth as SEQ ID NO: 1.

The present invention provides a Cre variant having Cre recombinase activity comprising amino acids in the sequence set forth as SEQ ID NO: 5, wherein the sequence of amino acids of the Cre variant is other than the sequence set forth as SEQ ID NO: 1.

The present invention provides a Cre variant having Cre recombinase activity comprising amino acids in the sequence set forth as SEQ ID NO: 7, wherein the sequence of amino acids of the Cre variant is other than the sequence set forth as SEQ ID NO: 1.

The present invention provides a polynucleotide encoding a Cre variant of the present invention.

The present invention provides an expression vector comprising a polynucleotide of the present invention operably linked to a promoter.

The present invention provides a recombinant virus comprising an expression vector of the present invention.

The present invention provides a cell comprising an expression vector of the present invention.

The present invention provides a non-human animal comprising a cell of the present invention.

The present invention provides a polynucleotide that is untranslated when expressed in a cell, comprising nucleotides in a sequence that is at least 80% identical to the nucleotide sequence set forth as SEQ ID NO: 4.

The present invention provides a polynucleotide that is untranslated when expressed in a cell, comprising nucleotides at least 80% identical to the nucleotide sequence set forth as SEQ ID NO: 6.

The present invention provides a polynucleotide that is untranslated when expressed in a cell, comprising nucleotides at least 80% identical to the nucleotide sequence set forth as SEQ ID NO: 8.

The present invention provides a isolated polypeptide comprising a first portion contiguous with a second portion, wherein the amino acid sequence of the first portion is less than about 90% identical to the sequence of amino acids 1-20 set forth in SEQ ID NO: 1, and the amino acid sequence of the second portion is at least about 90% identical to SEQ ID NO: 7.

The present invention provides a FLP variant having FLP recombinase activity comprising amino acids in the sequence set forth as SEQ ID NO: 17, and having other than a methionine at its N-terminus.

The present invention provides a polynucleotide that is untranslated when expressed in a cell, comprising nucleotides in a sequence that is at least 80% identical to the nucleotide sequence set forth as SEQ ID NO: 18.

The present invention provides a polynucleotide that is untranslated when expressed in a cell, comprising nucleotides in a sequence that is at least 80% identical to the nucleotide sequence set forth as SEQ ID NO: 22.

The present invention provides an expression vector comprising the polynucleotide, having other than in-frame nucleotides in a sequence encoding a start codon between the 5′end of the polynucleotide and any promoter within the expression vector within the expression vector.

The present invention provides a PhiC31 variant having PhiC31 recombinase activity comprising amino acids in the sequence set forth as SEQ ID NO: 21, and having other than a methionine at its 5′ end.

The present invention provides a polynucleotide that is untranslated when expressed in a cell, comprising nucleotides in a sequence that is at least 80% identical to the nucleotide sequence set forth as SEQ ID NO: 22.

The present invention provides an expression vector comprising the polynucleotide, having other than in-frame nucleotides in a sequence encoding a start codon between the 5′end of the polynucleotide and any promoter within the expression vector within the expression vector.

### BRIEF DESCRIPTION OF THE FIGURES

FIG. 1. Design of a trans-splicing ribozyme coupling Cre expression to the expression of the D2R receptor. (A) The trans-splicing is a bimolecular reaction overall strategy in which a ribozyme splices into an mRNA target such as the D2R. The ribozyme construct includes the Cre open reading frame (ORF), but in the absence of trans-splicing the ribozyme does not produce Cre protein because Cre has been engineered to remove all possible start codons. (B) The first step is the specific recognition of the ribozyme with the target. Specificity is achieved by Watson-Crick base pairing at (1) the internal guide sequence (IGS), which binds to the complementary sequence in the target; and (2) the extended guide sequence (EGS) which provides additional stability and specificity. (C) The trans-splicing reaction adds a start codon to the Cre ORF. (D) During translation, a virally-derived cis-acting hydrolase element (CHYSEL) 2a sequence co-translationally self-cleaves near the junction of the endogenous gene and the transgene; and (4) a sequence encoding Cre recombinase.

FIG. 2. Confirmation of trans-splicing in culture. HEK293 cells were co-transfected with either a target and the ribozyme-Cre (1) or separately transfected and co-plated (2). RT-PCR using primers for the product expected from successful trans-splicing of Cre into the target show a band at the expected size (˜120 bp), confirmed by sequencing. No band was observed in the negative control.

FIG. 3. Silent Cre Recombinase.

### DETAILED DESCRIPTION OF THE INVENTION

The present invention provides a trans-splicing ribozyme comprising:
i) a targeting nucleotide sequence that is complementary to a target nucleotide sequence within a mRNA that is expressed in a cell; contiguous with
ii) a catalytic RNA sequence; contiguous with
iii) a donor transcript, which donor transcript comprises at least a nucleotide sequence that encodes a trans-activator,
   wherein when the trans-splicing ribozyme is expressed in a cell, the catalytic RNA sequence cleaves the mRNA and ligates the donor transcript to the mRNA to generate a spliced mRNA which comprises the donor transcript, such that the donor transcript is translated as part of the spliced mRNA in the cell.

In some embodiments, the donor transcript further comprises a nucleotide sequence that encodes a protein cleavage sequence, wherein in the donor transcript the protein cleavage sequence is encoded before the trans-activator in the donor transcript, such that an polypeptide comprising the trans-activator is post-translationally or co-translationally cleaved from the polypeptide sequence that is translated or is being translated from the spliced mRNA.

In some embodiments, the protein cleavage sequence is co-translationally cleaved in the cell.

In some embodiments, the protein cleavage sequence is a cis-acting hydrolase element.

In some embodiments, the cis-acting hydrolase element is a virally derived cis-acting hydrolase element.

In some embodiments, the virally derived cis-acting hydrolase element is a CHYSEL 2a sequence.

In some embodiments, the mRNA is specifically expressed in one cell-type or a sub-type of cells.

In some embodiments, the mRNA encodes the D2R receptor.

In some embodiments, the targeting nucleotide sequence comprises an internal guide sequence (IGS) that is complementary to at least a portion of the target nucleotide sequence.

In some embodiments, the target nucleotide sequence of the mRNA to which the IGS is complementary is immediately followed by a uracil (U), and the targeting nucleotide sequence contains a guanine (G) following the IGS at a nucleotide position that forms a wobble base-pair with the U when the targeting nucleotide is bound to its complementary mRNA sequence.

In some embodiments, the IGS is at least 6 nucleotides in length.

In some embodiments, the IGS is 6 nucleotides in length.

In some embodiments, the IGS is fully complementary to at least a portion of the target sequence to which it is complementary.

In some embodiments, the IGS is complementary to the entire target nucleotide sequence.

In some embodiments, the IGS is fully complementary to the entire target nucleotide sequence.

In some embodiments, the targeting nucleotide sequence further comprises an extended guide sequence (EGS) that is complementary to a portion of the target nucleotide sequence that immediately follows the U that forms a wobble base-pair with the G that follows the IGS when the targeting nucleotide sequence is bound to its complementary target nucleotide within the mRNA.

In some embodiments, the EGS is 5-200 nucleotides in length.

In some embodiments, the EGS is 100 nucleotides in length.

In some embodiments, the EGS is fully complementary to the portion of the target nucleotide sequence to which it is complementary.

In some embodiments, the catalytic RNA sequence is about 400 nucleotides in length.

In some embodiments, the catalytic RNA sequence is derived from a Group I intron.

In some embodiments, the catalytic RNA sequence is derived from a Group II intron.

In some embodiments, the catalytic RNA sequence is derived from a ribozyme found in nature.

In some embodiments, the catalytic RNA sequence is derived from an artificial ribozyme.

In some embodiments, the catalytic artificial ribozyme is designed by an in vitro selection method.

In some embodiments, the in vitro selection method is SELEX.

In some embodiments, the catalytic RNA sequence that is capable of splicing the donor transcript to the mRNA at a position within the target nucleotide sequence is derived from the Group I intron of Tetrahymena thermophila.

In some embodiments, the nucleotide sequence of the donor transcript that encodes the trans-activator comprises a coding sequence for the trans-activator that lacks a translational start codon.

In some embodiments, the trans-activator is a tetracycline transactivator (tTA or tTA1.1), GAL4, or a recombinase. In some embodiments the tetracycline transactivator is tTA. In some embodiments the tetracycline transactivator is tTA1.1.

In some embodiments, the trans-activator is other than a recombinase.

In some embodiments, the trans-activator is a recombinase.

In some embodiments, the recombinase is Cre, FLP or PhiC31.

In some embodiments, the recombinase is Cre.

In some embodiments, the recombinase is silent Cre.

In some embodiments, the recombinase is FLP.

In some embodiments, the recombinase is silent FLP.

In some embodiments, the recombinase is PhiC31.

In some embodiments, the recombinase is silent PhiC31.

In some embodiments, the silent Cre is Cre1.25, Cre1.5 or Cre1.75.

In some embodiments, the silent Cre is a Cre variant of the invention.

In some embodiments, the silent FLP is FLP1.1.

In some embodiments, the silent FLP is a FLP variant of the invention.

In some embodiments, the silent PhiC31 is PhiC311.1.

In some embodiments, the silent PhiC31 is a PhiC31 variant of the invention.

The present invention provides a polynucleotide encoding a trans-splicing ribozyme the invention.

In some embodiments, the polynucleotide is about 1,500 nucleotides in length.

In some embodiments, the polynucleotide is in a vector that is suitable for viral delivery.

The present invention provides a virus comprising a polynucleotide of the invention.

In some embodiments, the virus is a recombinant adeno-associated virus (rAAV) or a recombinant lentivirus.

The present invention provides a cell comprising a polynucleotide of the invention.

The present invention provides non-human animal comprising the polynucleotide of the present invention.

In some embodiments, the polynucleotide is an isolated polynucleotide.

The present invention provides an expression vector comprising a polynucleotide of the invention operably linked to a promoter.

In some embodiments, the promoter is an RNA polymerase promoter.

In some embodiments, the expression vector is designed for delivery into cells by a virus.

The present invention provides a method of producing the trans-splicing ribozyme of the invention, comprising viral delivery of an expression vector comprising a polynucleotide encoding the trans-splicing ribozyme into a cell under conditions such that the cell expresses the trans-splicing ribozyme, thereby producing the trans-splicing ribozyme.

The present invention provides a method of expressing a recombinase-dependent transgene in a cell, comprising delivery of
a) a first expression vector which is an expression vector of the invention; and
b) a second expression vector which comprises the recombinase-dependent transgene,
   into the cell under conditions such that the cell expresses the trans-splicing ribozyme encoded in the first expression vector, and the trans-activator encoded by the trans-splicing ribozyme activates expression of the trans-activator-dependent transgene in the second expression vector, thereby expressing the trans-activator-dependent transgene in the cell.

In some embodiments, the first expression vector is delivered into the cell with a recombinant virus.

In some embodiments, the second expression vector is delivered into the cell with a recombinant virus.

In some embodiments, the cell is in an animal.

In some embodiments, the animal is a mammal.

In some embodiments, the mammal is a human.

In some embodiments, the mRNA containing the target nucleotide sequence is specifically expressed in the cell-type or cell sub-type to which the cell belongs, such that the trans-activator-dependent transgene is expressed in a cell-type or cell sub-type specific manner.

In some embodiments, the trans-activator is a recombinase.

In some embodiments, the recombinase is Cre.

In some embodiments, the recombinase is silent Cre.

In some embodiments, the silent Cre is Cre1.25, Cre1.5 or Cre1.75.

In some embodiments, the silent Cre is a Cre variant of the invention.

In some embodiments, when delivered into the cell, the recombinase-dependent transgene in the second expression vector contains a transcriptional stop cassette flanked by loxP recombination sequences, such that in the cell Cre or silent Cre removes the transcriptional stop cassette from the recombinase-dependent transgene and operably links a promoter of the second expression vector to the transgene.

In some embodiments, the recombinase is FLP.

In some embodiments, the recombinase is silent FLP.

In some embodiments, the silent FLP is FLP1.1.

In some embodiments, the silent FLP is a FLP variant of the invention.

In some embodiments, when delivered into the cell, the recombinase-dependent transgene in the second expression vector contains a transcriptional stop cassette flanked by FRT recombination sequences, such that in the cell FLP removes the transcriptional stop cassette from the recombinase-dependent transgene and operably links a promoter of the second expression vector to the transgene.

In some embodiments, the recombinase is PhiC31.

In some embodiments, the recombinase is silent PhiC31.

In some embodiments, the silent PhiC31 is PhiC311.1.

In some embodiments, the silent PhiC31 is a PhiC31 variant of the invention.

In some embodiments, when delivered into the cell, the recombinase-dependent transgene in the second expression vector contains a transcriptional stop cassette flanked by attB and attP recombination sequences, such that in the cell PhiC31 or silent PhiC31 removes the transcriptional stop cassette from the recombinase-dependent transgene and operably links a promoter of the second expression vector to the transgene.

In some embodiments, the promoter is constitutively active in the cell.

The present invention provides a method of expressing a trans-activator-dependent transgene in a cell containing a trans-activator-dependent transgene, comprising delivery of an expression vector of the present invention into the cell under conditions such that the cell expresses the trans-splicing ribozyme encoded in the expression vector, and the trans-activator encoded by the trans-splicing ribozyme activates expression of the trans-activator-dependent transgene in the cell, thereby expressing the trans-activator-dependent transgene in the cell.

In some embodiments, the expression vector is delivered into the cell with a recombinant virus.

In some embodiments, the cell is in an animal.

In some embodiments, the animal is a mammal.

In some embodiments, the mammal is a human.

In some embodiments, the mRNA molecule containing the target sequence is specifically expressed in the cell-type or cell sub-type to which the cell belongs, such that the recombinase-dependent transgene is expressed in a cell-type or cell sub-type specific manner.

In some embodiments, the trans-activator is a recombinase.

In some embodiments, the recombinase is Cre.

In some embodiments, the recombinase is silent Cre.

In some embodiments, the silent Cre is Cre1.25, Cre1.5 or Cre1.75.

In some embodiments, the silent Cre is a Cre variant of the invention.

In some embodiments, before delivery of the expression vector into the cell, the recombinase-dependent transgene contains a transcriptional stop cassette flanked by loxP recombination sequences, such that in the cell Cre or silent Cre removes the transcriptional stop cassette from the recombinase-dependent transgene and operably links a promoter to the transgene.

In some embodiments, the recombinase is FLP.

In some embodiments, the recombinase is silent FLP.

In some embodiments, the silent FLP is FLP1.1.

In some embodiments, the silent FLP is a FLP variant of the present invention.

In some embodiments, before delivery of the expression vector into the cell, the recombinase-dependent transgene contains a transcriptional stop cassette flanked by FRT recombination sequences, such that in the cell FLP or silent FLP removes the transcriptional stop cassette from the recombinase-dependent transgene and operably links a promoter to the transgene.

In some embodiments, the recombinase is PhiC31.

In some embodiments, the recombinase is silent PhiC31.

In some embodiments, the silent PhiC31 is PhiC311.1.

In some embodiments, the silent PhiC31 is a PhiC31 variant of the invention.

In some embodiments, before delivery of the expression vector into the cell, the recombinase-dependent transgene contains a transcriptional stop cassette flanked by attB and attP recombination sequences, such that in the cell PhiC31 or silent PhiC31 removes the transcriptional stop cassette from the recombinase-dependent transgene and operably links a promoter to the transgene.

In some embodiments, the promoter is constitutively active in the cell.

The present invention provides a Cre variant having Cre recombinase activity comprising amino acids in the sequence set forth as SEQ ID NO: 3, wherein the sequence of amino acids of the Cre variant is other than the sequence set forth as SEQ ID NO: 1.

The present invention provides a Cre variant having Cre recombinase activity comprising amino acids in the sequence set forth as SEQ ID NO: 5, wherein the sequence of amino acids of the Cre variant is other than the sequence set forth as SEQ ID NO: 1.

The present invention provides a Cre variant having Cre recombinase activity comprising amino acids in the sequence set forth as SEQ ID NO: 7, wherein the sequence of amino acids of the Cre variant is other than the sequence set forth as SEQ ID NO: 1.

In some embodiments, the Cre variant consists of amino acids in the sequence set forth as SEQ ID NO: 9.

In some embodiments, the Cre variant consists of amino acids in the sequence set forth as SEQ ID NO: 11.

In some embodiments, the Cre variant consists of amino acids in the sequence set forth as SEQ ID NO: 13.

In some embodiments, the Cre variant has substantially the same level of recombinase activity as Cre recombinase having the amino acid sequence set forth as SEQ ID NO: 1.

The present invention provides a polynucleotide encoding a Cre variant of the present invention.

The present invention provides an expression vector comprising a polynucleotide of the present invention operably linked to a promoter.

The present invention provides a recombinant virus comprising an expression vector of the present invention.

The present invention provides a cell comprising an expression vector of the present invention.

The present invention provides a non-human animal comprising a cell of the present invention.

The present invention provides a polynucleotide that is untranslated when expressed in a cell, comprising nucleotides in a sequence that is at least 80% identical to the nucleotide sequence set forth as SEQ ID NO: 4.

The present invention provides a polynucleotide that is untranslated when expressed in a cell, comprising nucleotides at least 80% identical to the nucleotide sequence set forth as SEQ ID NO: 6.

The present invention provides a polynucleotide that is untranslated when expressed in a cell, comprising nucleotides at least 80% identical to the nucleotide sequence set forth as SEQ ID NO: 8.

The present invention provides an expression vector comprising a polynucleotide of the invention, having other than in-frame nucleotides in a sequence encoding a start codon between the 5′end of the polynucleotide and any promoter within the expression vector within the expression vector.

The present invention provides a recombinant virus comprising an expression vector of the present invention.

The present invention provides a cell comprising an expression vector of the present invention.

The present invention provides a non-human animal comprising a cell of the present invention.

The present invention provides a isolated polypeptide comprising a first portion contiguous with a second portion, wherein the amino acid sequence of the first portion is less than about 90% identical to the sequence of amino acids 1-20 set forth in SEQ ID NO: 1, and the amino acid sequence of the second portion is at least about 90% identical to SEQ ID NO: 7.

In some embodiments, the amino acid sequence of the first portion is less than about 75% identical to the sequence of amino acids 1-20 set forth in SEQ ID NO: 1.

In some embodiments, the amino acid sequence of the first portion is less than about 50% identical to the sequence of amino acids 1-20 set forth in SEQ ID NO: 1.

In some embodiments, the amino acid sequence of the first portion is less than about 25% identical to the sequence of amino acids 1-20 set forth in SEQ ID NO: 1.

In some embodiments, the amino acid sequence of the first portion is less than about 0% identical to the sequence of amino acids 1-20 set forth in SEQ ID NO: 1.

In some embodiments, the amino acid sequence of the second portion is at least about 95% identical to SEQ ID NO: 7.

In some embodiments, the amino acid sequence of the second portion is at least about 99% identical to SEQ ID NO: 7.

The present invention provides a FLP variant having FLP recombinase activity comprising amino acids in the sequence set forth as SEQ ID NO: 17, and having other than a methionine at its N-terminus.

In some embodiments, the FLP variant consists of amino acids in the sequence set forth as SEQ ID NO: 17.

In some embodiments, the FLP variant has substantially the same level of recombinase activity as unmodified FLP recombinase having the amino acid sequence set forth as SEQ ID NO: 15.

The present invention provides a polynucleotide encoding a FLP variant of the present invention.

The present invention provides an expression vector comprising the polynucleotide operably linked to a promoter.

The present invention provides a recombinant virus comprising the expression vector.

The present invention provides a cell comprising the expression vector.

The present invention provides a non-human animal comprising the cell.

The present invention provides a polynucleotide that is untranslated when expressed in a cell, comprising nucleotides in a sequence that is at least 80 identical to the nucleotide sequence set forth as SEQ ID NO: 18.

The present invention provides an expression vector comprising the polynucleotide of the invention, having other than in-frame nucleotides in a sequence encoding a start codon between the 5′end of the polynucleotide and any promoter within the expression vector.

The present invention provides a recombinant virus comprising the expression vector.

The present invention provides a cell comprising the expression vector.

The present invention provides a non-human animal comprising the cell.

The present invention provides a PhiC31 variant having PhiC31 recombinase activity comprising amino acids in the sequence set forth as SEQ ID NO: 21, and having other than a methionine at its 5′ end.

In some embodiments, the PhiC31 variant consists essentially of amino acids in the sequence set forth as SEQ ID NO: 21.

In some embodiments, the PhiC31 variant has substantially the same level of recombinase activity as unmodified PhiC31 recombinase having the amino acid sequence set forth as SEQ ID NO: 19.

The present invention provides a polynucleotide encoding a PhiC31 variant of the present invention.

The present invention provides a expression vector comprising the polynucleotide operably linked to a promoter.

The present invention provides a recombinant virus comprising the expression vector.

The present invention provides a cell comprising the expression vector.

The present invention provides a non-human animal comprising the cell.

The present invention provides a polynucleotide that is untranslated when expressed in a cell, comprising nucleotides in a sequence that is at least 80% identical to the nucleotide sequence set forth as SEQ ID NO: 22.

The present invention provides an expression vector comprising the polynucleotide, having other than in-frame nucleotides in a sequence encoding a start codon between the 5′end of the polynucleotide and any promoter within the expression vector within the expression vector.

The present invention provides a recombinant virus comprising the expression vector.

The present invention provides a cell comprising the expression vector.

The present invention provides a non-human animal comprising the cell.

In some embodiments, the polynucleotide is an isolated polynucleotide.

Each embodiment disclosed herein is contemplated as being applicable to each of the other disclosed embodiments. Thus, all combinations of the various elements described herein are within the scope of the invention.

It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

### KEY TO THE SEQUENCE LISTING

### SEQ ID NO:1 Cre Amino Acid Sequence

### SEQ ID NO:2 Cre DNA Coding Sequence

### SEQ ID NO:3 Cre1.25 Amino Acid Sequence

### SEQ ID NO:4 Cre1.25 DNA Coding Sequence

### SEQ ID NO:5 Cre1.5 Amino Acid Sequence

### SEQ ID NO:6 Cre1.5 DNA Coding Sequence

### SEQ ID NO:7 Cre1.75 Amino Acid Sequence

### SEQ ID NO:8 Cre1.75 DNA Coding Sequence

### SEQ ID NO:9 CreM1.25 Amino Acid Sequence

### SEQ ID NO:10 CreM1.25 DNA Coding Sequence

### SEQ ID NO:11 CreM1.5 Amino Acid Sequence

### SEQ ID NO:12 CreM1.5 DNA Coding Sequence

### SEQ ID NO:13 CreM1.75 Amino Acid Sequence

### SEQ ID NO:14 CreM1.75 DNA Coding Sequence

### SEQ ID NO:15 FLP Amino Acid Sequence

### SEQ ID NO:16 FLP DNA Coding Sequence

SEQ ID NO:17 FLP1.1 Amino Acid Sequence (FLPe lacking an N-terminal methionine)
SEQ ID NO:18 FLP1.1 DNA Coding Sequence (FLPe lacking a start codon)

### SEQ ID NO:19 PhiC31 Amino Acid Sequence

### SEQ ID NO:20 PhiC31 DNA Coding Sequence

SEQ ID NO:21 PhiC311.1 Amino Acid Sequence (PhiC31o lacking an N-terminal methionine)
SEQ ID NO:22 PhiC311.1 DNA Coding Sequence (PhiC31o lacking a start codon)

### SEQ ID NO:23 tTA Amino Acid Sequence

### SEQ ID NO:24 tTA Coding Sequence

SEQ ID NO:25 tTA1.1 Amino Acid Sequence (tTA lacking an N-terminal methionine)
SEQ ID NO:26 tTA1.1 DNA Coding Sequence (tTA lacking a start codon)
SEQ ID NO:27 rtTA Amino Acid Sequence
SEQ ID NO:28 rtTA DNA Coding Sequence
SEQ ID NO:29 rtTA1.1 Amino Acid Sequence (lacking an N-terminal methionine)
SEQ ID NO:30 rtTA1.1 DNA Coding Sequence (lacking a start codon)

### SEQ ID NO:31 L21 Ribozyme DNA Coding Sequence

The sequences provided for FLP and PhiC31 in the sequence listing are for optimized FLP and PhiC31.

### TERMS

Unless otherwise defined, all technical and scientific terms used herein have the same meaning as commonly understood by a person of ordinary skill in the art to which this invention belongs.

As used herein, and unless stated otherwise or required otherwise by context, each of the following terms shall have the definition set forth below.

As used herein, “about” in the context of a numerical value or range means±10% of the numerical value or range recited or claimed, unless the context requires a more limited range.

As used herein, the term “sequence” may mean either a strand or part of a strand of nucleotides, or the order of nucleotides within a strand or part of a strand, depending on the appropriate context in which the term is used. Unless specified otherwise in context, the order of nucleotides is recited from the 5′ to the 3′ direction of a strand.

As used herein, the term “fully complementary” with regard to a sequence refers to a complement of the sequence by Watson-Crick base pairing, whereby guanine (G) pairs with cytosine (C), and adenine (A) pairs with either uracil (U) or thymine (T). A sequence may be fully complementary to the entire length of another sequence, or it may be fully complementary to a specified portion or length of another sequence. One of skill in the art will recognize that U may be present in RNA, and that T may be present in DNA. Therefore, an A within either of a RNA or DNA sequence may pair with a U in a RNA sequence or T in a DNA sequence.

As used herein, the term “wobble base pairing” with regard to two complementary nucleic acid sequences refers to the base pairing of G to uracil U rather than C, when one or both of the nucleic acid strands contains the ribonucleobase U.

The term “mRNA” refers to a nucleic acid transcribed from a gene from which a polypeptide is translated, and may include non-translated regions such as a 5′UTR and/or a 3′UTR. It will be understood that a trans-splicing ribozyme of the invention may comprise a nucleotide sequence that is complementary to any sequence of an mRNA molecule, including translated regions, the 5′UTR, the 3′UTR, and sequences that include both a translated region and a portion of either 5′UTR or 3′UTR.

“Nucleic acid” refers to polynucleotides such as deoxyribonucleic acid (DNA) and ribonucleic acid (RNA). The term can include single-stranded and double-stranded polynucleotides.

“Operably linked” means that the coding sequence is linked to a regulatory sequence in a manner which allows expression of the coding sequence. Regulatory sequences include promoters, enhancers, and other expression control elements that are art-recognized and are selected to direct expression of the coding sequence.

A “transduced cell” is one that has been genetically modified. Genetic modification can be stable or transient. Methods of transduction (i.e., introducing vectors or constructs into cells) include, but are not limited to, liposome fusion (transposomes), viral infection, and routine nucleic acid transfection methods such as electroporation, calcium phosphate precipitation and microinjection. Successful transduction will have an intended effect in the transduced cell, such as gene expression, gene silencing, enhancing a gene target, or triggering target physiological event.

“Vector” refers to a vehicle for introducing a nucleic acid into a cell. Vectors include, but are not limited to, plasmids, phagemids, viruses, bacteria, and vehicles derived from viral or bacterial sources (Dassie et al., Nature Biotechnology 27, 839-846 (2009), Zhou and Rossi, Silence, 1:4 (2010), NcNamera et al., Nature Biotechnology 24, 1005-1015 (2006)).

A “plasmid” is a circular, double-stranded DNA molecule. A useful type of vector for use in the present invention is a viral vector, wherein heterologous DNA sequences are inserted into a viral genome that can be modified to delete one or more viral genes or parts thereof. Certain vectors are capable of autonomous replication in a host cell (e.g., vectors having an origin of replication that functions in the host cell). Other vectors can be stably integrated into the genome of a host cell, and are thereby replicated along with the host genome.

### Ribozymes

Ribozymes are RNA molecules with catalytic activity (Uhlmann et al., 1987, Tetrahedron. Lett. 215, 3539-3542). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences. Methods of designing and constructing ribozymes which can cleave other RNA molecules in trans in a highly sequence specific manner have been developed and described in the art. For example, the cleavage activity of ribozymes can be targeted to specific RNAs by engineering a discrete “hybridization” region into the ribozyme. The hybridization region contains a sequence complementary to the target RNA and thus specifically hybridizes with the target RNA.

Specific ribozyme cleavage sites within an RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate RNA targets also can be evaluated by testing accessibility to hybridization with complementary oligonucleotides using ribonuclease protection assays. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Methods such as but not limited to viral delivery, microinjection, liposome-mediated transfection, electroporation, or calcium phosphate precipitation, can be used to introduce a ribozyme-containing DNA construct into cells. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or VAS element, and a transcriptional terminator signal, for controlling transcription of ribozymes in the cells (U.S. Pat. No. 5,641,673). Ribozymes also can be engineered to provide an additional level of regulation, so that destruction of mRNA occurs only when both a ribozyme and a target gene are induced in the cells.

### Ribozyme-Mediated Trans-Splicing

Aspect of the present invention relate to ribozyme-mediated RNA trans-splicing. Ribozymes may be engineered to join (i.e. trans-splice) part of an endogenous “target” mRNA transcript to another “donor” transcript. For example, a ribozyme may recognize a target via a complementary sequence. In the case of trans-splicing, the ribozyme is engineered to contain a sequence complementary to the target gene at the desired splice position. After recognizing its target, the ribozyme cleaves the target and ligates the donor transcript into the target transcript allowing translation of donor mRNA into a functional protein.

Aspects of the present invention exploit the high specificity of ribozyme-mediated trans-splicing. The specificity of trans-splicing has been demonstrated in experiments with Diphtheria toxin A (DTA), a potent cytotoxin. When a trans-splicing ribozyme encoding DTA is targeted to a particular target mRNA, cells expressing the target are killed with high efficiency. This demonstrates the very high specificity of the trans-splicing reaction, since even a very low level of off-target DTA trans-splicing would reduce cell viability. Similarly, trans-splicing ribozymes have been engineered to correct mutations in beta globulin transcripts responsible for sickle cell anemia and were able to discriminate mRNAs differing by only a single base (wt vs. mutant). Thus trans-splicing has the potential to provide the specificity needed to target trans-activators such as the recombinases Cre and FLP in a cell-type specific manner. See, e.g., Kohler et al. (1999) “Trans-splicing Ribozymes for Targeted Gene Delivery” J. Mol. Biol. 285, 1935-1950, the entire contents of which are incorporated herein by reference.

It will be understood that virtually any Group I catalytic intron can be adapted for use in embodiments of the subject application. Non-limiting examples of Group I catalytic introns that may be useful in, or that may be adapted for use in, embodiments of the present invention are described in Nielsen H, Johansen S D (2009). “Group I introns: Moving in new directions”. RNA Biol 6 (4): 375-83; Cate J H, Gooding A R, Podell E et al. (September 1996). “Crystal structure of a group I ribozyme domain: principles of RNA packing”. Science 273 (5282): 1678-85; Cech T R (1990). “Self-splicing of group I introns”. Annu. Rev. Biochem. 59: 543-68; Woodson S A (June 2005). “Structure and assembly of group I introns”. Curr. Opin. Struct. Biol. 15 (3): 324-30; Steitz, T A; Steitz J A (1993). “A general two-metal-ion mechanism for catalytic RNA”. Proc Natl Acad Sci USA 90 (14): 6498-6502; Stahley, M R; Strobel S A (2006). “RNA splicing: group I intron crystal structures reveal the basis of splice site selection and metal ion catalysis”. Curr Opin Struct Biol 16 (3): 319-326; Golden B L, Gooding A R, Podell E R, Cech T R (1998). “A preorganized active site in the crystal structure of the Tetrahymena ribozyme”. Science 282 (5387): 259-64; Golden B L, Kim H, Chase E (2005). “Crystal structure of a phage Twort group I ribozyme-product complex”. Nat Struct Nol Biol 12 (1): 82-9; Guo F, Gooding A R, Cech T R (2004). “Structure of the Tetrahymena ribozyme: base triple sandwich and metal ion at the active site”. Mol Cell 16 (3): 351-62; Brion P, Westhof E (1997). “Hierarchy and dynamics of RNA folding”. Annu Rev Biophys Biomol Struct 26: 113-37; Edgell D R, Belfort M, Shub D A (October 2000). “Barriers to intron promiscuity in bacteria”. J. Bacteriol. 182 (19): 5281-9; Sandegren L, Sjoberg B M (May 2004). “Distribution, sequence homology, and homing of group I introns among T-even-like bacterlophages: evidence for recent transfer of old introns”. J. Biol. Chem. 279 (21): 22218-27; Bonocora R P, Shub D A (December 2004). “A self-splicing group I intron in DNA polymerase genes of T7-like bacteriophages”. J. Bacteriol. 186 (23): 8153-5; Chauhan, S; Caliskan G, Briber R M, Perez-Salas U, Rangan P, Thirumalai D, Woodson S A (2005). “RNA tertiary interactions mediate native collapse of a bacterial group I ribozyme”. J Mol Biol 353 (5): 1199-1209; Haugen, P; Simon D M and Bhattacharya D (2005). “The natural history of group I introns”. TRENDS in Genetics 21 (2): 111-119; Rangan, P; Masquida, B, Westhof E, Woodson S A (2003). “Assembly of core helices and rapid tertiary folding of a small bacterial group I ribozyme”. Proc Natl Acad Sci USA 100 (4): 1574-1579; Schroeder, R; Barta A, Semrad K (2004). “Strategies for RNA folding and assembly”. Nat Rev Biol Cell Biol 5 (11): 908-919; Thirumalai, D; Lee N, Woodson S A, Klimov D (2001). “Early events in RNA folding”. Annu Rev Phys Chem 52: 751-762; and Lee C N, Lin J W, Weng S F, Tseng Y H (December 2009). “Genomic characterization of the intron-containing T7-like phage phiL7 of Xanthomonas campestris”. Appl. Environ. Microbiol. 75 (24): 7828-37, the entire contents of each of which are hereby incorporated herein by reference.

It will be understood that other catalytic RNA molecules can supply the catalytic RNA sequence of the subject application. Non-limiting examples include Group-II catalytic introns, and ribozymes that are designed by an In vitro selection method, such as Systematic Evolution of Ligands by Exponential Enrichment (SELEX).

Non-limiting examples of Group II catalytic introns that may be useful in, or that may be adapted for use in, embodiments of the present invention are described in Marcia M, Pyle A M. (2012) “Visualizing group II intron catalysis through the stages of splicing” Cell 151(3):497-507; de Lencastre A, Hamill S, Pyle A M (July 2005). “A single active-site region for a group II intron”. Nat. Struct. Nol. Biol. 12 (7): 626-7; Bonen, L; Vogel J (2001). “The ins and outs of group II introns”. Trends Genet 17 (6): 322-331; Chu, V T; Adamidi C, Liu Q, Perlman P S, Pyle A N (2001). “Control of branch-site choice by a group II intron”. EMBO J 20 (23): 6866-6876; Lehmann, K; Schmidt U (2003). “Group II introns: structure and catalytic versatility of large natural ribozymes”. Crit Rev Biochem Mol Biol 38 (3): 249-303; and Michel F, Umesono K, Ozeki H (October 1989). “Comparative and functional anatomy of group II catalytic introns—a review”. Gene 82 (1): 5-30, the entire contents of each of which are hereby incorporated herein by reference.

SELEX, which may be useful for obtaining catalytic RNA sequences that are useful in embodiments of the present invention, is described in Agresti et al. (2005) “Selection of ribozymes that catalyse multiple-turnover Diels-Alder cycloadditions by using in vitro compartmentalization” PNAS vol. 102 no. 45 16170-16175; Breaker and Joyce (July 1994) “Inventing and improving ribozyme function: rational design versus iterative selection methods” Cell Press, Trends in Biotechnology, Volume 12, Issue 7, Pages 268-275; Klug and Famulok (1994) “All you wanted to know about SELEX” Molecular Biology Reports, Volume 20, Issue 2, pp 97-107; Kawazoe et al. (2001) “In vitro selection of normatural ribozyme-catalyzing porphyrin metalation” Biomacromolecules, 2 (3), pp 681-686; and Levine H A, Nilsen-Hamilton N (2007). “A mathematical analysis of SELEX”. Computational biology and chemistry 31 (1): 11-35, the entire contents of each of which are hereby incorporated herein by reference.

### Use of Recombinases for Cell-Type Specific Expression

Recombinases are enzymes which catalyze the recombination of DNA between pairs of specific DNA sequences called recombination sites. Several recombinases have been used in mammals, including Cre and FLP. In both Cre and FLP, the recombination sites consist of specific sequences of 34 nucleotides (called loxP and FRT sites, for Cre and FLP, respectively). FRT and loxP sequences are very different; Cre does not act at FRT sites, nor does FLP act at loxP sites. Recombinases can be used to render the expression of transgenes of interest conditional upon their presence by means of a transcriptional “stop” cassette flanked by recombination sites placed between the promoter and the transgene. The stop cassette prevents transgene expression unless it is excised by the recombinase, in which case the transgene is expressed. Conditional expression can also be achieved by flip-excision (FLEX). Transgenes are delivered either by local injection of a recombinant virus such as recombinant AAV, or by breeding the recombinase knock-in mouse with a mouse in which the expression of the transgene is dependent on the recombinase. The progeny of such a cross express the transgene only in the cell population of interest. Transgene expression thus depends on the logical AND of the recombinase and the appropriately engineered transgene with properly placed recombination sites.

Breaking the problem into two components-cell-type specific recombinase expression and recombinase-dependent transgene expression—has two advantages compared with expressing the transgene directly from the locus of the endogenous gene (e.g. expressing a transgene directly under the control of the endogenous promoter). First, because the recombinase acts as a switch, the expression level of the transgene is decoupled from the expression level of the endogenous gene for which it is a marker; expression of the recombinase need only surpass the threshold sufficient to activate the switch, and the expression of the transgene can be driven by a strong promoter. Thus robust expression of a transgene coupled to a particular promoter can be achieved, even in the case where a promoter is only weakly active. The second advantage is combinatorial: there is no need to generate separate constructs for each combination of expression pattern and transgene, since novel combinations can be produced by combining recombinases (activators) and recombination-dependent transgenes (effectors). Thus N recombinase-dependent transgene and K recombinase constructs can yield potentially N×K distinct transgene expression profiles. The use of recombinases reduces the number of constructs needed to at most N+K instead of N×K.

### Trans-Activators

Artificial trans-activation of a gene may be achieved with a trans-activator gene and a region of DNA according to methods that are well known in the art of molecular biology. The trans-activator gene expresses a trans-activator which can interact with the region of DNA to activate a trans-activator-dependent gene. For example, a trans-activator may be a transcription factor that binds to specific promoter region of DNA to activate the expression of a gene that is operably linked to the specific promoter region of DNA. In some embodiments, the expression of one trans-activator can activate multiple trans-activator-dependent genes that are operably linked to the specific promoter region.

Aspects of the present invention relate to a trans-splicing ribozyme comprising a trans-activator. In some embodiments, the trans-activator is a recombinase. In some embodiments the trans-activator is other than a recombinase. Non-limiting examples of trans-activators other than recombinases are the tetracycline transactivator (tTA) protein, which is useful in connection with Tetracycline-Controlled Transcriptional Activation System (TET system), and GAL4 which is useful in the GAL4-UAS system. Non-limiting examples of the TET system, which is useful in embodiments of the subject invention, is described in Bujard, Hermann; M. Gossen (1992). “Tight Control of Gene Expression in Mammalian Cells by Tetracycline-Responsive Promoters.”. Proc. Natl. Acad. Sci. U.S.A. 89 (12): 5547-51; Urlinger, Stefanie; Baron, Udo; Thellmann, Marion; Hasan, Mazahir T.; Bujard, Herman; Hillen, Wolfgang (2000). “Exploring the sequence space for tetracycline-dependent transcriptional activators: Novel mutations yield expanded range and sensitivity.”. Proc. Natl. Acad. Sci. U.S.A. 97 (14): 7963-8; and Zhou, X.; Vink, M.; Klave, B.; Berkhout, B.; Das, A. T. (2006). “Optimization of the Tet-On system for regulated gene expression through viral evolution.”. Gene Ther. 13 (19): 1382-1390, the entire contents of each of which are incorporated herein by reference. The GAL4-UAS system is discussed in Brand A H, Perrimon N. (Jun. 1, 1993). “Targeted gene expression as a means of altering cell fates and generating dominant phenotypes”. Development 118: 401-415; Duffy, J B. (2002). “GAL4 system in Drosophila: A fly geneticist's Swiss army knife.”. Genesis 32: 1-15; Janice A. Fischer, Edward Giniger, Tom Maniatis, and Mark Ptashne (1988). “GAL4 activates transcription in Drosophila”. Nature (6167): 853-6; Webster N, Jin J R, Green S, Hollis M, Chambon P. (1988). “The yeast UASG is a transcription enhancer in human HeLa cells in the presence of the GAL4 trans-activator”. Cell 52 (2): 169-78; Liu Y and Lehman M (2008). “A genomic response to the yeast transcription factor GAL4 in Drosophila”. Fly (Austin) 2 (2); Katharine O. Hartley, Stephen L. Nutt, and Enrique Amaya (2002). “Targeted gene expression in transgenic Xenopus using the binary Gal4-UAS system”. Proc Natl Acad Sci UAS 99 (3): 1377-82; Davison J M, Akitake C M, Goll M G, Rhee J M, Gosse N, Baier H, Halpern M E, Leach S D, Parsons M J (2007). “Transactivation from Gal4-VP16 transgenic insertions for tissue-specific cell labeling and ablation in zebrafish”. Developmental Biology 304 (2): 811-24; Suster, Maximiliano L and Seugnet, Laurent and Bate, Michael and Sokolowski, Marla B (2004). “Refining GAL4-driven transgene expression in Drosophila with a GAL80 enhancer-trap”. Genesis (Wiley Online Library) 39 (4): 240-245; and Luan, Haojiang and Peabody, Nathan C and Vinson, Charles R and White, Benjamin H (2006). “Refined spatial manipulation of neuronal function by combinatorial restriction of transgene expression”. Neuron (Elsevier) 52 (3): 425-436, the entire contents of each of which are incorporated herein by reference.

In some embodiments the trans-activator is the tetracycline transactivator (tTA) protein. In some embodiments, the trans-activator is GAL4.

### Non-Limiting Examples of Trans-Activator-Dependent Transgenes

Aspects of the invention relate to the expression and/or detection of a trans-activator-dependent transgene. In some embodiments, the trans-activator-dependent transgene is a recombinase-dependent transgene. It will be understood that virtually any gene conceivable can be a trans-activator-dependent transgene (e.g. endogenous, or exogenous, natural or synthetic). Additionally, any noncoding element that can be made conditional (e.g. RNAi, lncRNA, etc.) may be a trans-activator-dependent transgene. However, non-limiting examples of trans-activator dependent transgenes are provided herein.

Aspects of the present invention may be used to introduce a second (or repaired) copy of a mutated gene, or a double expression of an existing gene in a cell-type specific way. Additionally, aspects of the present invention can be used to introduce DREADDS (Designer Receptors Exclusively Activated by Designer Drugs), Optogenetic probes, cytotoxins, full-length endogenous/exocenous genes from nature, other trans-activators or recombinases, etc. Non-limiting examples of DREADDS that are useful in embodiments of the present invention are described in Rogan and Roth, Pharmacol Rev 2011 June; 63(2):291-315 and Dong et al, Mol Biosystems 2010 August; 6(8):1376-80, the entire contents of each of which are hereby incorporated herein by reference.

In some embodiments, the recombinase-dependent transgene is a reporter polypeptide. A reporter polypeptide may be used to specifically label a cell type or cell sub-type. The reporter polypeptide may be an epitope tag, a fluorescent protein, a luminescent protein, a chromogenic enzyme, streptavidin, beta-galactosidase, or any other reporter polypeptide disclosed herein or known in the art.

Examples of epitope tags include but are not limited to V5-tag, Myc-tag, HA-tag, FLAG-tag, GST-tag, and His-tags. Additional examples of epitope tags are described in the following references: Huang and Honda, CED: a conformational epitope database. BMC Immunology 7:7 www.biomedcentral.com/1471-2172/7/78B1. Retrieved Feb. 16, 2011 (2006); and Walker and Rapley, Molecular biomethods handbook. Pg. 467 (Humana Press, 2008). These references in their entireties are hereby incorporated by reference into this application. In some embodiments of the invention a label comprising an antibody or an antibody fragment is used to detect the localization and/or expression of a fusion protein which comprises an epitope tag.

Fluorescent proteins will be well known to one skilled in the art, and include but are not limited to green fluorescent protein (GFP), enhanced green fluorescent protein (EGFP), Renilla Reniformis green fluorescent protein, GFPmut2, GFPuv4, yellow fluorescent protein (YFP), such as VENUS, enhanced yellow fluorescent protein (EYFP), cyan fluorescent protein (CFP), enhanced cyan fluorescent protein (ECFP), blue fluorescent protein (BFP), enhanced blue fluorescent protein (EBFP), citrine and red fluorescent protein from discosoma (dsRED), AcGFP, TagGFP, EBFP2, Asurite, mCFP, mKeima-Red, Azami Green, YagYFP, Topaz, mCitrine, Kusabira Orange, mOrange, mKO, TagRFP, RFP, DsRed2, mStrawberry, mRFP1, mCherry, and mRaspberry.

Examples of luminescent proteins include but are not limited to enzymes which may catalyze a reaction that emits light, such as luciferase. Examples of chromogenic enzymes include but are not limited to horseradish peroxidase and alkaline phosphatase.

Additional, non-limiting examples of suitable detectable reporter polypeptides include chloramphenicol acetyltransferase (CAT), luminescent proteins such as luciferase lacZ (β-galactosidase) and horseradish peroxidase (HRP), nopaline synthase (NOS), octopine synthase (OCS), and alkaline phosphatase.

In some embodiments, the recombinase-dependent transgene can be separately introduced into the cell harboring the transplicing ribozyme construct (e.g., co-transfected, etc.). In some embodiments, the recombinase-dependent transgene can be on the transplicing ribozyme construct, and the marker gene expression can be controlled by the same or a separate translation unit, for example, by an IRES (internal ribosomal entry site).

Reporters polypeptides can also be those that confer resistance to a drug, such as neomycin, ampicillin, bleomycin, chloramphenicol, gentamycin, hygromycin, kanamycin, lincomycin, methotrexate, phosphinothricin, puromycin, doxycycline, and tetracyclin. Recombinase-dependent transgenes can also be lethal genes, such as herpes simplex virus-thymidine kinase (HSV-TK) sequences, as well as sequences encoding various toxins including the diphtheria toxin, the tetanus toxin, the cholera toxin and the pertussis toxin. A further negative selection marker is the hypoxanthine-guanine phosphoribosyl transferase (HPRT) gene for negative selection in 6-thioguanine.

Reporter polypeptides may be detected indirectly or directly. General techniques and compositions for detecting and/or observing and/or analyzing reporter polypeptides and other transgenes which are useful in the present invention are described in the following references: Tsien et al., Fluorophores for confocal microscopy. Handbook of biological confocal microscopy. New York: Plenum Press, 1995; Rietdorf, Mocroscopic techniques. Advances in Biochemical Engineering/Biotechnology. Berlin: Springer 2005; Lakowicz, J R, Principles of fluorescence spectroscopy (3^{rd} ed.). Springer, 2006. These references in their entireties are hereby incorporated by reference into this application.

### Optogenetic Probes

In some embodiments, the recombinase-dependent transgene encodes an optogenetic probe. In response to light, optogenetic probes influence the behavior of cells expressing them. For example, a neuron expressing an optogenetic probe may be stimulated with light. See Witten et al. (2010) “Cholinergic interneurons control local circuit activity and cocaine conditioning” Science 330 (6011): 1677-81, PMC 3142356, the entire contents of which are incorporated herein by reference.

Non-limiting examples of optogenetic probes include fusion proteins comprising opsins and G-protein receptors (such as chimeric rhodopsin containing the beta 2-adrenergic receptor cytoplasmic loops), and optically controlled GTPases and adenylyl cyclases (such as in Phy-KrasCAAX PIF-YFP recruitment pair systems, forms of Racl fused to the photoreactive LOV (light oxygen voltage) domain from phototropin (e.g. PA-Racl-T17N), photoactivated adenylyl cyclase (bPAC)), BlaC, and BlgC. Exemplary optogenetic probes and methods that are useful in embodiments of the present invention are described in: Kim et al. (2005) “Light-driven activation of beta 2-adrenergic receptor signaling by a chimeric rhodopsin containing the beta 2-adrenergic receptor cytoplasmic loops” Biochemistry 44 (7): 2284-92 PMID 15709741; Airan et al. (2009) “Temporally precise in vivo control of intracellular signalling” Nature 458 (7241): 1025-9. PMID 19295515; (2009) Levskaya et al. (2009) “Spatiotemporal control of cell signalling using a light-switchable protein interaction” Nature 461 (7266): 997-1001 PMID 19749742; Wu et al. (2009) “A genetically encoded photoactivatable Rac controls the motility of living cells” Nature 461 (7260): 104-8 PMID 19693014, PMC 2766670; Yazawa et al. (2009) “Induction of protein-protein interactions in live cells using light” Nature Biotechnology 27 (10): 941-5 PMID 19801976; Stierl et al. (2011) “Light modulation of cellular cAMP by a small bacterial photoactivated adenylyl cyclase, bPAC, of the soil bacterium Beggiatoa” J. Biol. Chem. 286 (2): 1181-8 PHC 3020725, PMID 21030594; and Ryu et al. (2010) “Natural and engineered photoactivated nucleotidyl cyclases for optogenetic applications” J. Biol. Chem. 285 (53): 41501-8 PMC 3009876, PMID 21030591, the entire contents of each of which are incorporated by reference.

### RNA Interference

In some embodiments the recombinase-dependent transgene encodes an interfering RNA (RNAi) molecule. RNAi involves mRNA degradation, but many of the biochemical mechanisms underlying this interference are unknown. The use of RNAi has been described in Fire et al., 1998, Carthew et al., 2001, and Elbashir et al., 2001, the contents of which are incorporated herein by reference.

Interfering RNA or small inhibitory RNA (RNAi) molecules include short interfering RNAs (siRNAs), repeat-associated siRNAs (rasiRNAs), and micro-RNAs (miRNAs) in all stages of processing, including shRNAs, pri-miRNAs, and pre-miRNAs. These molecules have different origins: siRNAs are processed from double-stranded precursors (dsRNAs) with two distinct strands of base-paired RNA; siRNAs that are derived from repetitive sequences in the genome are called rasiRNAs; miRNAs are derived from a single transcript that forms base-paired hairpins. Base pairing of siRNAs and miRNAs can be perfect (i.e., fully complementary) or imperfect, including bulges in the duplex region.

Interfering RNA molecules encoded by recombinase-dependent transgenes of the invention can be based on existing shRNA, siRNA, piwi-interacting RNA (piRNA), micro RNA (miRNA), double-stranded RNA (dsRNA), antisense RNA, or any other RNA species that can be cleaved inside a cell to form interfering RNAs, with compatible modifications described herein.

As used herein, an “shRNA molecule” includes a conventional stem-loop shRNA, which forms a precursor miRNA (pre-miRNA). “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. When transcribed, a shRNA may form a primary miRNA (pri-miRNA) or a structure very similar to a natural pri-miRNA. The pri-miRNA is subsequently processed by Drosha and its cofactors into pre-miRNA. Therefore, the term “shRNA” includes pri-miRNA (shRNA-mir) molecules and pre-miRNA molecules.

A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). The terms “hairpin” and “fold-back” structures are also used herein to refer to stem-loop structures. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches.

“RNAi-expressing construct” or “RNAi construct” is a generic term that includes nucleic acid preparations designed to achieve an RNA interference effect. An RNAi-expressing construct comprises an RNAi molecule that can be cleaved in vivo to form an siRNA or a mature shRNA. For example, an RNAi construct is an expression vector capable of giving rise to an siRNA or a mature shRNA in vivo. Non-limiting examples of vectors that may be used in accordance with the present invention are described herein and will be well known to a person having ordinary skill in the art. Exemplary methods of making and delivering long or short RNAi constructs can be found, for example, in WO01/68836 and WO01/75164.

### Use of RNAi

RNAi is a powerful tool for in vitro and in vivo studies of gene function in mammalian cells and for therapy in both human and veterinary contexts. Inhibition of a target gene is sequence-specific in that gene sequences corresponding to a portion of the RNAi sequence, and the target gene itself, are specifically targeted for genetic inhibition. Three mechanisms of utilizing RNAi in mammalian cells have been described. The first is cytoplasmic delivery of siRNA molecules, which are either chemically synthesized or generated by DICER-digestion of dsRNA. These siRNAs are introduced into cells using standard transfection methods. The siRNAs enter the RISC to silence target mRNA expression.

The second mechanism is nuclear delivery, via viral vectors, of gene expression cassettes expressing a short hairpin RNA (shRNA). The shRNA is modeled on micro interfering RNA (miRNA), an endogenous trigger of the RNAi pathway (Lu et al., 2005, Advances in Genetics 54: 117-142, Fewell et al., 2006, Drug Discovery Today 11: 975-982). Conventional shRNAs, which mimic pre-miRNA, are transcribed by RNA Polymerase II or III as single-stranded molecules that form stem-loop structures. Once produced, they exit the nucleus, are cleaved by DICER, and enter the RISC as siRNAs.

The third mechanism is identical to the second mechanism, except that the shRNA is modeled on primary miRNA (shRNAmir), rather than pre-miRNA transcripts (Fewell et al., 2006). An example is the miR-30 miRNA construct. The use of this transcript produces a more physiological shRNA that reduces toxic effects. The shRNAmir is first cleaved to produce shRNA, and then cleaved again by DICER to produce siRNA. The siRNA is then incorporated into the RISC for target mRNA degradation. However, aspects of the present invention relate to RNAi molecules that do not require DICER cleavage. See, e.g., U.S. Pat. No. 8,273,871, the entire contents of which are incorporated herein by reference.

For mRNA degradation, translational repression, or deadenylation, mature miRNAs or siRNAs are loaded into the RNA Induced Silencing Complex (RISC) by the RISC-loading complex (RLC). Subsequently, the guide strand leads the RISC to cognate target mRNAs in a sequence-specific manner and the Slicer component of RISC hydrolyses the phosphodiester bound coupling the target mRNA nucleotides paired to nucleotide 10 and 11 of the RNA guide strand. Slicer forms together with distinct classes of small RNAs the RNAi effector complex, which is the core of RISC. Therefore, the “guide strand” is that portion of the double-stranded RNA that associates with RISC, as opposed to the “passenger strand,” which is not associated with RISC.

It is not necessary that there be perfect correspondence of the sequences, but the correspondence must be sufficient to enable the RNA to direct RNAi inhibition by cleavage or blocking expression of the target mRNA. In preferred RNA molecules, the number of nucleotides which is complementary to a target sequence is 16 to 29, 18 to 23, or 21-23, or 15, 16, 17, 18, 19, 20, 21, 22, 23, 24, or 25.

### Vectors

In certain embodiments, expression vectors encoding a trans-splicing ribozyme or a recombinase-dependent transgene may be based on CMV-based or MSCV-based vector backbones. In certain embodiments, expression vectors may be based on self-inactivating lentivirus (SIN) vector backbones. Non-limiting examples of vector backbones and methodologies for construction of expression vectors suitable for use in connection with the subject application, and methods for introducing such expression vectors into various mammalian cells are found in the following references: Premsrurit P K. et al., Cell, 145(1):145-158, 2011, Gottwein E. and Cullen B. Meth. Enzymol. 427:229-243, 2007, Dickens et al., Nature Genetics, 39:914-921, 2007, Chen et al., Science 303: 83-86, 2004; Zeng and Cullen, RNA 9: 112-123, 2003, the contents of which are specifically incorporated herein by reference.

The vectors described in International application no. PCT/US2008/081193 (WO 09/055,724) and methods of making and using the vectors are incorporated herein by reference. The disclosure provided therein illustrates the general principles of vector construction and expression of sequences from vector constructs, and is not meant to limit the present invention.

Trans-splicing ribozymes and recombinase-dependent transgenes can be expressed from vectors in almost any cell type. In a certain embodiment, the vector is a viral vector. Exemplary viral vectors include retroviral, including lentiviral, adenoviral, baculoviral and avian viral vectors.

Retroviruses from which the retroviral plasmid vectors can be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus. A retroviral plasmid vector can be employed to transduce packaging cell lines to form producer cell lines. Examples of packaging cells which can be transfected include, but are not limited to, the PE501, PA317, R-2, R-AM, PA12, T19-14x, VT-19-17-H2, RCRE, RCRIP, GP+E-86, GP+envAm12, and DAN cell lines as described in Miller, Human Gene Therapy 1:5-14 (1990), which is incorporated herein by reference in its entirety. The vector can transduce the packaging cells through any means known in the art. A producer cell line generates infectious retroviral vector particles which include polynucleotide encoding a DNA replication protein. Such retroviral vector particles then can be employed, to transduce eukaryotic cells, either in vitro or in vivo. The transduced eukaryotic cells will express a DNA replication protein.

In certain embodiments, cells can be engineered using an adeno-associated virus (AAV). AAVs are naturally occurring defective viruses that require helper viruses to produce infectious particles (Muzyczka, N., Curr. Topics in Microbiol. Immunol. 158:97 (1992)). It is also one of the few viruses that can integrate its DNA into nondividing cells. Vectors containing as little as 300 base pairs of AAV can be packaged and can integrate, but space for exogenous DNA is limited to about 4.5 kb. Methods for producing and using such AAVs are known in the art. See, for example, U.S. Pat. Nos. 5,139,941, 5,173,414, 5,354,678, 5,436,146, 5,474,935, 5,478,745, and 5,589,377. For example, an AAV vector can include all the sequences necessary for DNA replication, encapsidation, and host-cell integration. The recombinant AAV vector can be transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the polynucleotide construct. These viral particles are then used to transduce eukaryotic cells.

In certain embodiments, cells can be engineered using a lentivirus and lentivirus based vectors. Such an approach is advantageous in that it allows for tissue-specific expression in animals through use of cell type-specific pol II promoters, efficient transduction of a broad range of cell types, including nondividing cells and cells that are hard to infect by retroviruses, and inducible and reversible gene knockdown by use of tet-responsive and other inducible promoters. Efficient production of replication-incompetent recombinant lentivirus may be achieved, for example, by co-transfection of expression vectors and packaging plasmids using commercially available packaging cell lines, such as TLA-HEK293™, and packaging plasmids, available from Thermo Scientific/Open Biosystems, Huntsville, Ala.

Essentially any method for introducing a nucleic acid construct into cells can be employed. Physical methods of introducing nucleic acids include injection of a solution containing the construct, bombardment by particles covered by the construct, soaking a cell, tissue sample or organism in a solution of the nucleic acid, or electroporation of cell membranes in the presence of the construct. A viral construct packaged into a viral particle can be used to accomplish both efficient introduction of an expression construct into the cell and transcription of the encoded trans-splicing ribozyme or recombinase-dependent transgene. Other methods known in the art for introducing nucleic acids to cells can be used, such as lipid-mediated carrier transport, chemical mediated transport, such as calcium phosphate, and the like.

Examples of useful promoters in the context of the invention are tetracycline-inducible promoters (including TRE-tight), IPTG-inducible promoters, tetracycline transactivator systems, and reverse tetracycline transactivator (rtTA) systems. Constitutive promoters can also be used, as can cell- or tissue-specific promoters. Many promoters will be ubiquitous, such that they are expressed in all cell and tissue types. A certain embodiment uses tetracycline-responsive promoters, one of the most effective conditional gene expression systems in in vitro and in vivo studies.

Expression vectors of the present invention may contain regulatory sequences such as transcription control sequences, translation control sequences, origins of replication, and other regulatory sequences are compatible with the recombinant cell and that control the expression of nucleic acid molecules of the present invention. In particular, recombinant molecules of the present invention include transcription control sequences. Transcription control sequences are sequences which control the initiation, elongation and termination of transcription. Particularly important transcription control sequences are those which control transcription initiation, such as promoter, enhancer, operator and repressor sequences.

All publications and other references mentioned herein are incorporated by reference in their entirety, as if each individual publication or reference were specifically and individually indicated to be incorporated by reference. Publications and references cited herein are not admitted to be prior art.

This invention will be better understood by reference to the Experimental Details which follow, but those skilled in the art will readily appreciate that the specific experiments detailed are only illustrative of the invention as defined in the claims which follow thereafter.

### EXPERIMENTAL DETAILS

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### Example 1

### Trans-Splicing Ribosymes

### Strategy for Achieving Specific Recombinase Expression

FIG. 1 summarizes an exemplary strategy for using ribozyme-mediated trans-splicing to couple an mRNA encoding Cre into the mRNA of an endogenous target gene such as the D2R receptor. (In what follows Cre is used as an example; the approach is exactly analogous for other recombinases, such as FLP).

To limit expression specifically to D2R expressing neurons, the IGS and EGS sequences are engineered to target complementary sequences in the mRNA encoding the D2R transcript. The engineered ribozyme contains all of the necessary coding sequence of Cre, but is missing a translational start codon. In the absence of its target the ribozyme is expressed but not translated; only upon trans-splicing into the appropriate target does it acquire the start codon necessary for translation. The resultant transcript includes a portion of the target gene, but this is co-translationally cleaved at a virally-derived cis-acting hydrolase element (CHYSEL) 2a sequence included in the ribozyme. The result is that the expression of functional Cre protein is conditional on the presence of a D2R mRNA. Initial experiments in cultured non-neuronal cells confirm trans-splicing (FIG. 2).

The construct shown in FIG. 1, which consists of the Cre open reading frame (1 kb), the CHYSEL sequence (66 bp), the trans-splicing ribozyme (˜400 bp), and the target antisense (˜100 bp), is short enough (˜1.5 kb) that it can be readily delivered using recombinant AAV. Thus if a recombinant virus is used to infect a heterogeneous population of cells, of which only some express the D2R receptor, the expression of Cre will be restricted to the D2R subpopulation. If these cells are co-infected with a rAAV expressing a Cre-dependent transgene such as GFP, then only those cells expressing the target D2R will express GFP. This approach can be used to target any gene expressed in the brain or other tissues.

### Example 2

### Silent Cre Recombinase

CreM (CreM1, Cre1.25, CreM1.5, or CreM1.75) plasmid was co-transfected into HEK293 cells with a reporter plasmid the constitutively expresses mCherry and that expresses GFP in a Cre-dependent fashion (FIG. 3A). Cells were incubated for 48 hrs at 37° C. and then harvested for flow cytometry analysis (an example of which is shown in FIG. 3B). Cells which expressed mCherry (positive for transfection) were further assayed for GPP expression. CreM1 is functional without a start codon. CreM2 shows little activity with or without a start codon. CreM1.25, CreM1.5, and CreM1.75 showed the desired activity pattern; recombination is detected only when a start codon is provided.

### Example 3

### General Method for Targeting Genes to Specific Nearonal Subtypes in Mammals

The ability to manipulate gene expression in genetically defined neuronal subtypes provides a powerful tool for dissecting neural circuits. A general method for achieving cell-type specific expression in the mammalian nervous system would represent an important advance. The ideal approach to achieving cell-type specific expression would combine the specificity of knock-in transgenics with the convenience of viral delivery and would open the door to cell-type specific expression of transgenes in many model organisms (namely rats and primates). Such a technique is developed based on ribozyme (catalytic RNA) mediated RNA trans-splicing (joining of two separate mRNA transcripts).

The group I intron from Tetrahymena thermophila is a catalytic RNA (or ribozyme) with the remarkable ability to perform a cleavage-ligation reaction in the absence of proteins. Though its normal activity is to splice itself out of an mRNA transcript (joining the preceding and following segments into an uninterrupted transcript), it is possible to engineer the ribozyme to trans-splice (join part of one “donor” transcript to another “target” transcript). The tetrahymena group I intron recognizes its target via a 6 bp complementary sequence known as the internal guide sequence (IGS). In the case of trans-splicing, the ribozyme is engineered to contain an IGS complementary to the target gene at the desired splice position. Additional specificity is achieved by adding a second complementary region (complementary to the target mRNA) known as the extended guide sequence (EGS). After recognizing its target, the ribozyme cleaves the target and ligates its cargo (the donor transcript) into the target transcript allowing translation of donor mRNA into a functional protein. This reaction has been shown to be specific enough to trans-splice cytotoxins into target transcripts (Ayre et. al., 1999) and to preferentially splice into a desired target over an undesired target that differed by a single nucleotide (Byun et. al. 2003). Thus, an approach herein is to couple the mRNA transcript of a transgene to the mRNA transcript of a cell-specific endogenous gene (i.e. somatostatin).

The central technical challenges confronted are efficiency and specificity of ribozyme-mediated trans-splicing. Low efficiency presents a significant problem for transgenes like GFP and Channelrhodopsin-2 (ChR2), which require relatively high levels of expression to be useful. To overcome this challenge the cre-lox system is adopted. First, trans-splicing is used to splice Cre into a target gene (such as somatostatin), thereby achieving cell-type specific expression of Cre. Then a transgene, which is rendered conditional on the presence of Cre (Kuhlman and Huang, 2008), as delivered in conjunction with the ribozyme. Cre recombination is a highly efficient reaction and even low levels of Cre expression are sufficient to mediate recombination between loxP sites (and thereby activate the conditional transgene). Low specificity is a larger concern as it increases the false positive rate and thus may lead to failure of the technology. However, high degrees of specificity can be achieved, based on the findings of other groups (Ayre et. al. 1999, and Byun et. al., 2003). Additionally, an alternative strategy (Strategy 2) circumvents this problem and provide other advantages in its own right.

### Strategy 1

### Optimize the Specificity of Trans-Placing in Cell Culture

Several ribozymes are constructed varying critical parameters (including the length and thermodynamic stability (Herschlag et. al., 1991) of the IGS and EGS regions) to increase the specificity of the ribozyme for the target transcript (somatostatin). The target (somatostatin), ribozyme (somatostatin-ribozyme carrying Cre), and Cre-dependent transgene (GFP) are co-transfected into HEK293 cells. Specificity is assayed with fluorescence microscopy. To discriminate between non-specific splicing and leaky expression of Cre, a mutant ribozyme lacking catalytic activity is designed. Functional versions of Cre are generated in which potential start codons have been replaced in order to reduce the possibility of leaky expression of unspliced Cre. If nonspecific splicing does occur 5′ Rapid Amplification of cDNA Ends (RACE) is used to determine the identity of the nonspecific targets. This aids in designing more specific ribozymes.

### Validating the Technology In Vivo

A sufficient level of specificity is achieved in vitro. AAV based viruses of Ribozyme-Cre and the Cre-dependent transgene are generated. Cell culture work is built upon and somatostatin interneurons are targeted. The efficiency and specificity of the virus is validated with immunohistology.

### Strategy 2

### Engineer a System with Feedback

A system in which the expression level of the transgene can be more carefully controlled is generated. Thus a similar system that relies on the Tet-On system is designed. In this scheme, the reverse tetracycline trans activator (rtTA) is spliced into an endogenous gene (i.e. somatostatin), thus creating cell type-specific expression of rtTA. The transcription of the transgene expressed (i.e. GFP) is driven off of a minimal promoter flanked by the tet responsive element. When rtTA binds the tet responsive element, transcription of the transgene takes place. The ability of rtTA to bind the tetracycline responsive element is dependent on an additional external variable—the amount of doxycycline. Thus, by administering differing amounts of doxycycline a gain is effectively put on the system. This can be used to overcome non-specific expression: a hundred fold or even twenty fold ratio of transgene expression in somatostatin positive vs. somatostatin negative cells can be exploited with a proper gain. Modulation of the expression of the transgene is also advantageous to avoid negative effects of over-expression, modulate the effects of the transgene (i.e. repeat experiment with varying amount of ChR2 present or turn ChR2 on/off), etc.

This approach is successful and the data herein is built upon to establish a ribozyme resource available to the scientific community at large, containing ribozymes targeting many other genes of interest. Comparable resources for shRNA-based knock-down of genes have proven useful in neurobiology and other fields.

### Discussion

A core idea underlying the approaches herein is to use ribozyme-mediated trans-splicing to couple an mRNA encoding a recombinase such as Cre or FLP into the mRNA of an endogenous gene. The expression of the recombinase can then be used to switch on expression of an exogenous transgene.

### Ribozyme-Mediated Trans-Splicing.

An ideal approach for achieving cell-type specific expression would combine the specificity of knock-in transgenics with the convenience of viral delivery. The present invention provides such a technique based on ribozyme-mediated RNA trans-splicing. The key to the approach herein is that instead of coupling expression of the transgene to the promoter driving the endogenous gene of interest, methods of the present invention move a step downstream, and couple the translation of the transgene directly to the mRNA transcript encoding the endogenous gene.

Trans-splicing ribozymes provided in embodiments of the invention are derived from the group I intron from Tetrahymena thermophile, a catalytic RNA (or ribozyme) with the ability to perform a cleavage-ligation reaction in the absence of proteins. Though its normal activity is to splice itself out of an mRNA transcript (joining the preceding and following segments into an uninterrupted transcript), it is possible to engineer the ribozyme to transsplice (join part of one “donor” transcript to another “target” transcript). The tetrahymena group I intron recognizes its target via a 6 bp complementary sequence known as the internal guide sequence (IGS). In the case of trans-splicing, the ribozyme is engineered to contain an IGS complementary to the target gene at the desired splice position. The only absolute requirement for splicing is an available uracil ‘U’ in the target mRNA. Additional specificity is achieved by adding a second complementary region (complementary to the target mRNA) known as the extended guide sequence (EGS). After recognizing its target, the ribozyme cleaves the target and ligates its cargo (the donor transcript) into the target transcript allowing translation of donor mRNA into a functional protein.

### Silent Cre Recombinase

Any approach that requires Cre not be active when its coding sequence (CDS) is out of frame with respect to a desired start codon requires minimal leak of translated Cre. Because Cre is an amplifier, even low levels of leak at the level of translation will activate a Cre-dependent transgene. Unexpectedly, initial experiments revealed that functional Cre protein was produced even in the absence of the first ATG (start codon). Herein, it was hypothesized that Cre translation was initiating downstream of the first ATG, perhaps at the second ATG. To test this hypothesis a construct termed CreM2S (Cre initiating on the second Methionine) was made, which was an N-terminal truncation of Cre, starting at the second in frame ATG. This construct failed to express a functional Cre. Thus it was reasoned that Cre translation initiates somewhere between the first and second ATG sequence. Using a binomial search algorithm, a series of truncated Cre sequences were designed to identify a sequence in which the expression of functional Cre required the addition of an ATG start codon. Three truncations, CreM1.25, CreM1.5, and CreM1.75, showed little or no activity in the absence of a start codon but were fully active upon addition of an in-frame ATG (FIG. 3).

### REFERENCES

1. Ayre, B. G., Kohler, U., Goodman, H. M., Haseloff, J. Design of highly specific cytotoxins by using trans-splicing ribozymes. PNAS 96, 3507-3512 (1999).
2. Byun, J. et al. Efficient and specific repair of sickle B-globin RNA by trans-splicing ribozymes. RNA 1254-1263 (2003).
3. Cech, T. Self-splicing of group I introns. Annual review of biochemistry (1990).
4. Herschlag, D. Implications of ribozyme kinetics for targeting the cleavage of specific RNA molecules in vivo: more isn't always better. PNAS 88, 6921-5 (1991).
5. Inoue, T., Sullivan, F. X. 6 Cech, T. R. Intermolecular exon ligation of the rRNA precursor of Tetrahymena: oligonucleotides can function as 5′ exons. Cell 43, 431-7 (1985).
6. Kuhlman, S. J. & Huang, Z. J. High-resolution labeling and functional manipulation of specific neuron types in mouse brain by Cre-activated viral gene expression. PloS one 3, e2005 (2008).

1. A trans-splicing ribozyme comprising:
i) a targeting nucleotide sequence that is complementary to a target nucleotide sequence within a mRNA that is expressed in a cell; contiguous with
ii) a catalytic RNA sequence; contiguous with
iii) a donor transcript, which donor transcript comprises at least a nucleotide sequence that encodes a trans-activator,
wherein when the trans-splicing ribozyme is expressed in a cell, the catalytic RNA sequence cleaves the mRNA and ligates the donor transcript to the mRNA to generate a spliced mRNA which comprises the donor transcript, such that the donor transcript is translated as part of the spliced mRNA in the cell.

2. The trans-splicing ribozyme of claim 1, wherein the donor transcript further comprises a nucleotide sequence that encodes a protein cleavage sequence, wherein in the donor transcript the protein cleavage sequence is encoded before the trans-activator in the donor transcript, such that an polypeptide comprising the trans-activator is post-translationally or co-translationally cleaved from the polypeptide sequence that is translated or is being translated from the spliced mRNA.

3. The trans-splicing ribozyme of claim 1, wherein the protein cleavage sequence is co-translationally cleaved in the cell.

4. The trans-splicing ribozyme of claim 1, wherein the protein cleavage sequence is a cis-acting hydrolase element.

5. (canceled)

6. (canceled)

7. The trans-splicing ribozyme of claim 1, wherein the mRNA is specifically expressed in one cell-type or a sub-type of cells.

8. The trans-splicing ribozyme of claim 7, wherein the mRNA encodes the D2R receptor.

9. The trans-splicing ribozyme of claim 1, wherein the targeting nucleotide sequence comprises an internal guide sequence (IGS) that is complementary to at least a portion of the target nucleotide sequence.

10. The trans-splicing ribozyme of claim 9, wherein the target nucleotide sequence of the mRNA to which the IGS is complementary is immediately followed by a uracil (U), and the targeting nucleotide sequence contains a guanine (G) following the IGS at a nucleotide position that forms a wobble base-pair with the U when the targeting nucleotide is bound to its complementary mRNA sequence.

11-15. (canceled)

16. The trans-splicing ribozyme of claim 9, wherein the targeting nucleotide sequence further comprises an extended guide sequence (EGS) that is complementary to a portion of the target nucleotide sequence that immediately follows the U that forms a wobble base-pair with the G that follows the IGS when the targeting nucleotide sequence is bound to its complementary target nucleotide within the mRNA.

17-20. (canceled)

21. The trans-splicing ribozyme of claim 1, wherein the catalytic RNA sequence is derived from a Group I intron.

22-27. (canceled)

28. The trans-splicing ribozyme of claim 1, wherein the nucleotide sequence of the donor transcript that encodes the trans-activator comprises a coding sequence for the trans-activator that lacks a translational start codon.

29. (canceled)

30. (canceled)

31. The method of claim 29, wherein the trans-activator is a recombinase.

32-44. (canceled)

45. A polynucleotide encoding the trans-splicing ribozyme of claim 1.

46. (canceled)

47. (canceled)

48. A virus comprising the polynucleotide of claim 45.

49. (canceled)

50. A cell comprising the polynucleotide of claim 45.

51. A non-human animal comprising the polynucleotide of claim 45.

52. (canceled)

53. An expression vector comprising the polynucleotide of claim 45 operably linked to a promoter.

54. (canceled)

55. (canceled)

56. A method of producing the trans-splicing ribozyme of claim 1, comprising viral delivery of an expression vector comprising a polynucleotide encoding the trans-splicing ribozyme into a cell under conditions such that the cell expresses the trans-splicing ribozyme, thereby producing the trans-splicing ribozyme.

57. A method of expressing a recombinase-dependent transgene in a cell, comprising delivery of
a) a first expression vector which is the expression vector of claim 53; and
b) a second expression vector which comprises the recombinase-dependent transgene,
into the cell under conditions such that the cell expresses the trans-splicing ribozyme encoded in the first expression vector, and the trans-activator encoded by the trans-splicing ribozyme activates expression of the trans-activator-dependent transgene in the second expression vector, thereby expressing the trans-activator-dependent transgene in the cell.

58-103. (canceled)

104. A Cre variant having Cre recombinase activity comprising amino acids in the sequence set forth as SEQ ID NO: 3, wherein the sequence of amino acids of the Cre variant is other than the sequence set forth as SEQ ID NO: 1.

105-156. (canceled)


SEQUENCE LISTING (31 sequences)

SEQ ID NO: 1 - protein, 343 aa, Bacteriophage P1
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 2 - DNA, 1032 bp, Bacteriophage P1
atgtccaatttactgactgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 3 - protein, 337 aa, Bacteriophage P1
VHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 4 - DNA, 1014 bp, Bacteriophage P1
gtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 5 - protein, 330 aa, Bacteriophage P1
LPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 6 - DNA, 993 bp, Bacteriophage P1
ttaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 7 - protein, 323 aa, Bacteriophage P1
DEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 8 - DNA, 972 bp, Bacteriophage P1
gatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 9 - protein, 338 aa, Artificial
  CreM1.25 Amino Acid Sequence
MVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 10 - DNA, 1017 bp, Artificial
  CreM1.25 DNA Coding Sequence
atggtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 11 - protein, 331 aa, Artificial
  CreM1.5 Amino Acid Sequence
MLPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 12 - DNA, 996 bp, Artificial
  CreM1.5 DNA Coding Sequence
atgttaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 13 - protein, 324 aa, Artificial
  CreM1.75 Amino Acid Sequence
MDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 14 - DNA, 975 bp, Artificial
  CreM1.75 DNA Coding Sequence
atggatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 15 - protein, 423 aa, Artificial
  FLP Amino Acid Sequence
MSQFDILCKTPPKVLVRQFVERFERPSGEKIASCAAELTYLCWMITHNGTAIKRATFMSYNTIISNSLSFDIVNKSLQFKYKTQKATILEASLKKLIPAWEFTIIPYNGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYPIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI

SEQ ID NO: 16 - DNA, 1272 bp, Artificial
  FLP DNA Coding Sequence
atgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctga

SEQ ID NO: 17 - protein, 422 aa, Artificial
  FLP1.1 Amino Acid Sequence (FLPe lacking an N-terminal methionine)
SQFDILCKTPPKVLVRQFVERFERPSGEKIASCAAELTYLCWMITHNGTAIKRATFMSYNTIISNSLSFDIVNKSLQFKYKTQKATILEASLKKLIPAWEFTIIPYNGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYPIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI

SEQ ID NO: 18 - DNA, 1269 bp, Artificial
  FLP1.1  DNA Coding Sequence (FLPe lacking a start codon
agccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctga

SEQ ID NO: 19 - protein, 612 aa, Artificial
  PhiC31 Amino Acid Sequence
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAAPKKKRKV

SEQ ID NO: 20 - DNA, 1839 bp, Artificial
  PhiC31 DNA Coding Sequence
atggatacctacgccggagcctacgacagacagagccgggagagagagaacagcagcgccgccagccccgccacccagagaagcgccaacgaggataaggccgccgatctgcagagagaggtggagagggacggcggcagattcagatttgtgggccacttcagcgaggcccctggcaccagcgccttcggcaccgccgagagacccgagttcgagagaatcctgaacgagtgtagggccggcaggctgaacatgatcatcgtgtacgacgtgtcccggttcagcaggctgaaggtgatggacgccatccctatcgtgtccgagctgctggccctgggcgtgaccatcgtgtccacccaggaaggcgtctttagacagggcaacgtgatggacctgatccacctgatcatgaggctggacgccagccacaaggagagcagcctgaagagcgccaagatcctggacaccaagaacctgcagagggagctgggcggctatgtgggcggcaaggccccctacggcttcgagctggtgtccgagaccaaggagatcacccggaacggcaggatggtgaacgtggtgatcaacaagctggcccacagcaccacccccctgaccggccccttcgagtttgagcccgacgtgatcaggtggtggtggcgggagatcaagacccacaagcacctgcctttcaagcccggcagccaggccgccatccaccccggcagcatcaccggcctgtgtaagagaatggacgccgacgccgtgcccaccagaggcgagaccatcggcaagaaaaccgccagcagcgcctgggaccccgccaccgtgatgagaatcctgagggaccctaggatcgccggcttcgccgccgaggtgatctacaagaagaagcccgacggcacccccaccaccaagatcgagggctacagaatccagagagaccccatcaccctgagacctgtggagctggactgtggccctatcatcgagcctgccgagtggtacgagctgcaggcctggctggacggcagaggcagaggcaagggcctgagcagaggccaggccatcctgagcgccatggacaagctgtactgtgagtgtggcgccgtgatgaccagcaagagaggcgaggagagcatcaaggacagctaccggtgccggagaagaaaggtggtggaccccagcgcccctggccagcacgagggcacctgtaatgtgagcatggccgccctggacaagttcgtggccgagcggatcttcaacaagatccggcacgccgagggcgacgaggagaccctggccctgctgtgggaggccgccagaagattcggcaagctgaccgaggcccccgagaagagcggcgagagggccaacctggtggccgagagagccgacgccctgaacgccctggaggagctgtacgaggacagagccgccggagcctatgacggccctgtgggcaggaagcacttcagaaagcagcaggccgccctgaccctgagacagcagggcgccgaggaaagactggccgagctggaggccgccgaggcccctaagctgcccctggatcagtggttccccgaggatgccgacgccgaccccaccggccccaagtcctggtggggcagagccagcgtggacgacaagagggtgttcgtgggcctgttcgtggataagatcgtggtgaccaagagcaccaccggcaggggccagggcacccccatcgagaagagagccagcatcacctgggccaagcctcccaccgacgacgacgaggatgacgcccaggacggcaccgaggacgtggccgcccctaagaaaaagcggaaagtgtga

SEQ ID NO: 21 - protein, 611 aa, Artificial
  PhiC311.1 Amino Acid Sequence (PhiC31o lacking an N-termina methionine)
DTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAAPKKKRKV

SEQ ID NO: 22 - DNA, 1836 bp, Artificial
  PhiC311.1 DNA Coding Sequence (PhiC31o lacking a start codon)
gatacctacgccggagcctacgacagacagagccgggagagagagaacagcagcgccgccagccccgccacccagagaagcgccaacgaggataaggccgccgatctgcagagagaggtggagagggacggcggcagattcagatttgtgggccacttcagcgaggcccctggcaccagcgccttcggcaccgccgagagacccgagttcgagagaatcctgaacgagtgtagggccggcaggctgaacatgatcatcgtgtacgacgtgtcccggttcagcaggctgaaggtgatggacgccatccctatcgtgtccgagctgctggccctgggcgtgaccatcgtgtccacccaggaaggcgtctttagacagggcaacgtgatggacctgatccacctgatcatgaggctggacgccagccacaaggagagcagcctgaagagcgccaagatcctggacaccaagaacctgcagagggagctgggcggctatgtgggcggcaaggccccctacggcttcgagctggtgtccgagaccaaggagatcacccggaacggcaggatggtgaacgtggtgatcaacaagctggcccacagcaccacccccctgaccggccccttcgagtttgagcccgacgtgatcaggtggtggtggcgggagatcaagacccacaagcacctgcctttcaagcccggcagccaggccgccatccaccccggcagcatcaccggcctgtgtaagagaatggacgccgacgccgtgcccaccagaggcgagaccatcggcaagaaaaccgccagcagcgcctgggaccccgccaccgtgatgagaatcctgagggaccctaggatcgccggcttcgccgccgaggtgatctacaagaagaagcccgacggcacccccaccaccaagatcgagggctacagaatccagagagaccccatcaccctgagacctgtggagctggactgtggccctatcatcgagcctgccgagtggtacgagctgcaggcctggctggacggcagaggcagaggcaagggcctgagcagaggccaggccatcctgagcgccatggacaagctgtactgtgagtgtggcgccgtgatgaccagcaagagaggcgaggagagcatcaaggacagctaccggtgccggagaagaaaggtggtggaccccagcgcccctggccagcacgagggcacctgtaatgtgagcatggccgccctggacaagttcgtggccgagcggatcttcaacaagatccggcacgccgagggcgacgaggagaccctggccctgctgtgggaggccgccagaagattcggcaagctgaccgaggcccccgagaagagcggcgagagggccaacctggtggccgagagagccgacgccctgaacgccctggaggagctgtacgaggacagagccgccggagcctatgacggccctgtgggcaggaagcacttcagaaagcagcaggccgccctgaccctgagacagcagggcgccgaggaaagactggccgagctggaggccgccgaggcccctaagctgcccctggatcagtggttccccgaggatgccgacgccgaccccaccggccccaagtcctggtggggcagagccagcgtggacgacaagagggtgttcgtgggcctgttcgtggataagatcgtggtgaccaagagcaccaccggcaggggccagggcacccccatcgagaagagagccagcatcacctgggccaagcctcccaccgacgacgacgaggatgacgcccaggacggcaccgaggacgtggccgcccctaagaaaaagcggaaagtgtga

SEQ ID NO: 23 - protein, 335 aa, Artificial
  tTA Amino Acid Sequence
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 24 - DNA, 1008 bp, Artificial
  tTA Coding Sequence
atgagtagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtag

SEQ ID NO: 25 - protein, 334 aa, Artificial
  tTA1.1 Amino Acid Sequence (tTA lacking an N-terminal methionine)
SRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 26 - DNA, 1005 bp, Artificial
  tTA1.1 DNA Coding Sequence (tTA lacking a start codon)
agtagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtag

SEQ ID NO: 27 - protein, 248 aa, Artificial
  rtTA Amino Acid Sequence
MSRLDKSKVINGALELLNGVGIEGLTTRKLAQKLGVEQPTLYWHVKNKRALLDALPIEMLDRHHTHFCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPTDALDDFDLDMLPADALDDFDLDMLPADALDDFDLDMLPG

SEQ ID NO: 28 - DNA, 747 bp, Artificial
  rtTA DNA Coding Sequence
atgtctagactggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaa

SEQ ID NO: 29 - protein, 247 aa, Artificial
  rtTA1.1 Amino Acid Sequence (lacking an N-terminal methionine)
SRLDKSKVINGALELLNGVGIEGLTTRKLAQKLGVEQPTLYWHVKNKRALLDALPIEMLDRHHTHFCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPTDALDDFDLDMLPADALDDFDLDMLPADALDDFDLDMLPG

SEQ ID NO: 30 - DNA, 744 bp, Artificial
  rtTA1.1 DNA Coding Sequence (lacking a start codon)
tctagactggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaa

SEQ ID NO: 31 - DNA, 393 bp, Artificial
  L21 Ribozyme DNA Coding Sequence
ggagggaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaatttgtatgcgaaagtatattgattagttttggagtactcg